(19) United States
(12) Patent Application Publication
(10) Pub. No.: US 2021/0100860 A1
(43) Pub. Date: Apr. 8, 2021

(54) USE OF COMPOSITIONS OF WATER/ALCOHOL EXTRACTS OF ANTRODIA CINNAMOMEA CUT-LOG WOOD CULTIVATED FRUITING BODY AND SOLID-STATE CULTIVATED MYCELIUM AS AUXILIARIES FOR ANTI-CANCER AGENTS

(71) Applicant: Taiwan Leader Biotech Corp., Taipei City (TW)

(72) Inventors: Chin-Chung LIN, Taipei City (TW); Chiu-Ping LO, Taipei City (TW); Yun-Yu CHEN, Taipei City (TW); Tzung-Hsien LAI, Taipei City (TW); Li-Chuan HSU, Taipei City (TW)

(21) Appl. No.: 17/082,876

(22) Filed: Oct. 28, 2020

Related U.S. Application Data
(63) Continuation of application No. 15/280,092, filed on Sep. 29, 2016.

(30) Foreign Application Priority Data
Jun. 23, 2016 (TW) 105119699

(51) Int. Cl. A61K 36/07; A61K 31/704; A61K 31/555; A61K 31/513; A61K 45/06

(52) U.S. Cl. CPC A61K 36/07; A61K 31/704; A61K 2236/333; A61K 31/513; A61K 45/06; A61K 31/555

(57) ABSTRACT

The present invention relates a use of Antrodia cinnamomea composition, consisting of 50-99% (W/W) of Antrodia cinnamomea solid-state cultivated mycelium water/alcohol extracts and 1-50% (W/W) of cut-log wood cultivated fruiting body water/alcohol extracts, in the preparation of auxiliary agents for chemotherapy. The Antrodia cinnamomea composition of the present invention has proven effects on improving the anti-cancer effects when combined with chemotherapy drugs, and reducing the toxicity and side effects caused by chemotherapy treatments. The composition of present invention can be applied to use as an auxiliary for anti-cancer agents.

### BACKGROUND OF THE INVENTION

### Technical Field of the Invention

The present invention relates a chemotherapy auxiliary agent comprising an Antrodia cinnamomea (also named Antrodia camphorata or Taiwanofungus camphoratus) composition. Especially, the present invention relates an A. cinnamomea composition for the preparation of auxiliaries of anti-cancer agents, consisting of 50-99% (W/W) of A. cinnamomea solid-state cultivated mycelium water/alcohol extracts and 1-50% (W/W) of cut-log wood cultivated fruiting body water/alcohol extracts.

### Background

Cancer is one of the diseases with highest death rate in the world. According to the statistical data in World Health Organization, it is shown that the number of global cancer occurrence was 14.09 million, and the number of death is 8.2 million in 2012. The incidence of cancer will increase due to gradual aging in demographic structure. It is estimated that the number of occurrence in 2030 will rise to 21.26 million, and the number of death will reach 12.66 million, increasing global cancer burden.

Currently, the main treatment of cancer is by the ways of surgery, radiotherapy and chemotherapy. However, there are many defects existed in the traditional cancer treatment methods. One of the defects is that only patients in early state can be treated with surgery, but metastasis may be happened no matter the surgery is success or failure.

Chemotherapy is a systemic treatment and can successfully reduce the loading of many solid tumors. The antineoplastic drugs used in the chemotherapy for eliminating the rapidly proliferating cells are lack of an ideal selectivity. Therefore, in the inhibition of cancer cells, the body proliferative cells such as bone marrow, gastrointestinal and reproductive cells and the central nervous system are frequently influenced, and some of the antineoplastic drugs will affect the function of liver, kidney, heart and the endocrine system. Especially, the drug resistance of tumor is considered the biggest obstacle to chemotherapy. In view of the side effects of chemotherapy to cause physical discomforts in cancer patients, thereby affecting whether cancer patients can continue to complete the entire course, therefore, the intervention of assistant agents during chemotherapy allows cancer patients keeping chemotherapy, reduces and improves the side effects caused by the chemotherapy.

Antrodia cinnamomea is a unique medicinal fungus in Taiwan. A. cinnamomea is a perennial fungus belonging to Aphyllophorales and Polyporaceae. Natives in Taiwan have used A. cinnamomea to relieve hangovers and alcohol-related symptoms. Among Taiwanese folk medicines, the fruiting body of A. cinnamomea is believed to be effective for inflammation, liver disease and gastrointestinal discomfort. Like the general edible and medicinal mushrooms, A. cinnamomea has many complex components with physiological activities, such as triterpenoids, polysaccharides, adenosine, vitamins, proteins, nucleic acids, steroids and others. Many studies have confirmed that A. cinnamomea has positive effects on anti-tumor, improving immunity, anti-allergy, anti-pathogen, anti-hypertension, lowering blood sugar and cholesterol, and is useful in liver protection and treatment of liver-related diseases.

Researches indicated that the extracts of fruiting body and mycelium of A. cinnamomea may have a function and capacity of free radical scavenging and anti-oxidation, reducing the alcohol-induced acute liver injury, protecting acute and chronic liver injury induced by carbon tetrachloride, enhancing immunity, and inhibiting tumor cell growth.

In recent years, the industry also performed a lot of researches in the artificial inoculation technology in the tree, Cinnamomum kanehirae Hayata cut-log wood cultivation. From the experimental results, it is showed that there is no significant difference between the growth of cultivated A. cinnamomea and natural A. cinnamomea, when using the success rate of inoculation and the fruiting body growth rate as indicators. That means the cut-log wood of Cinnamomum kanehirae Hayata can also be used in cultivating A. cinnamomea fruiting bodies of quite good qualities. However, the difference between cultivated and natural A. cinnamomea will be showed in a further analysis of the content and physical activity of their secondary metabolites. According to the experimental results, it is showed that a considerable amount of secondary metabolites can still be obtained from the cut-log wood cultivated A. cinnamomea even after three consecutive extractions. The result indicates that the cut-log wood cultivated A. cinnamomea possesses a higher content of secondary metabolites. In addition, the A. cinnamomea growing in different hosts also have quite different compositions of secondary metabolites. In Cinnamomum kanehirae Hayata cut-log wood cultivated A. cinnamomea, the content of low polarity secondary metabolites is much higher than in the A. cinnamomea grown from other species of trees. The secondary metabolites of these high contents usually exhibit the most obvious tumor suppressing activity.

There are many researches for the use of A. cinnamomea in cancer treatment, including the effects of A. cinnamomea extracts, isolates and ingredients contained therein on the inhibition of tumor cell growth. For example, Taiwan patent 1484954 disclosed an anti-cancer agent comprising 4-acetyl-antroquinonol B. Taiwan patent 1379678 disclosed a compound 4-hydroxy-2,3-dimethoxy-6-methyl-5-(3,7,11-trimethyl-2,6,10-dodecatriene)-2-cyclohexenone isolated from the A. cinnamomea extract, and compositions thereof used in inhibiting the growth of lymphoma tumor cells and gastric cancer cell TSGH-9201. Taiwan patent 1363631 disclosed a dehydrosulphurenic acid isolated from the A. cinnamomea extract, which is useful in inhibiting the growth of leukemia and pancreatic cancer cells. Patent publication 20081111 described a 4,7-dimethoxy-5-methyl-1,3-benzodioxole, which is useful in inhibiting the growth of breast cancer, liver cancer and prostate cancer cells. However, existing researches on the anti-cancer effects of A. cinnamomea mainly focused on the separate anti-cancer effects of A. cinnamomea fruiting body extract or mycelium extract, as well as the cancer cell proliferation inhibiting effects of the components contained therein.

CN 103300421 disclosed a medicated food combination for auxiliary cancer therapy containing A. cinnamomea mycelium combined with multiple medicinal herb extracts and targeted foods for advanced liver cancer, lung cancer, colon cancer, stomach cancer and acute and chronic leukemia patients, providing a decoction food to the adjunctive therapy in a diet therapeutic way. U.S. Pat. No. 9,044,467 B2 developed a liquid fermented A. cinnamomea, having effects of improving the side effects induced by platinum-based or anthracycline-based anti-cancer drugs, such as pain, fatigue, depression, and shortened effective time and physical decline. US patent application 20130089627 disclosed a method of administering an ethyl acetate extract of liquid fermented A. cinnamomea containing 4-acetyl-antroquinonol B to treat the cancers induced by cancer stem cells, and to increase the effects on inhibiting cancer cell proliferation when combined with a chemotherapy drug Cisplatin or Taxol, or in combination of radiation therapy.

Nevertheless, no combination of the water/alcohol extract of A. cinnamomea solid-state cultivated mycelium and cut-log wood cultivated fruiting body with certain proportion has been revealed to use as assistant agent for chemotherapy drugs in cancer inhibition and reducing side effects caused in the chemotherapy.

### SUMMARY OF INVENTION

In the present invention, it is found that an A. cinnamomea composition comprised of 50-99% (W/W) of A. cinnamomea solid-state cultivated mycelium water/alcohol extracts and 1-50% (W/W) of cut-log wood cultivated fruiting body water/alcohol extracts exhibits an assistant effect on anti-cancer drugs to improve the inhibition of cancer cell proliferation and attenuate the side effects caused by the drugs used in a chemotherapy, especially to reduce the reduction of macrophage colony (CFU-GM) numbers in bone marrow and the lowed numbers of white blood cells, red blood cells, lymphocytes and neutrophils caused by the administration of the chemotherapy drugs.

Accordingly, in one aspect, the present invention relates to an A. cinnamomea composition for using as an anti-cancer drug auxiliary, consisting of 50-99% (W/W) of A. cinnamomea solid-state cultivated mycelium water/alcohol extracts and 1-50% (W/W) of cut-log wood cultivated fruiting body water/alcohol extracts. Preferably, the A. cinnamomea composition is composed of 60-95% (W/W) of A. cinnamomea solid-state cultivated mycelium water/alcohol extracts and 5-40% (W/W) of cut-log wood cultivated fruiting body water/alcohol extracts.

In certain embodiments of the invention, the anti-cancer drug auxiliary is used to enhance the inhibitory effects of anti-cancer drugs on cancer cell proliferation. In other embodiments of the invention, the anti-cancer drug auxiliary is used to reduce the side effects caused by anti-cancer drugs.

In one embodiment of the invention, the anti-cancer drug auxiliary is used to attenuate the reduction of macrophage colony (CFU-GM) numbers in bone marrow caused by the administration of the chemotherapy drugs. In another embodiment of the invention, the anti-cancer drug auxiliary is used to improve and restore the lowed numbers of blood cells caused by the chemotherapy, including the number of white blood cells, red blood cells, lymphocytes and neutrophils.

In the present invention, the anti-cancer drug includes, but is not limited to, antimetabolites, alkylating agents, anthracyclines, antibiotics, antimitotic agents, proteasome inhibitors and platinum chemotherapy drugs. In preferable embodiments of the invention, the anti-cancer drug comprises, but is not limited to, 5-fluorouracil (5-FU), epirubicin, oxaliplatin or combinations thereof. In other embodiments of the invention, the cancer is selected from a group consisted of lung cancer, colon cancer, gastric cancer and breast cancer, and preferably is a gastric cancer.

### BRIEF DESCRIPTION OF THE DRAWINGS

FIG. 1 shows effects of the oral administration of A. cinnamomea compositions with various combinations of A. cinnamomea solid-state cultivated mycelium water/alcohol extracts (SC) and cut-log wood cultivated fruiting body water/alcohol extracts (FB) on the tumor weight in MKN-45 gastric tumor-bearing mice.

FIG. 2 shows the auxiliary effects of A. cinnamomea composition (L) of solid-state cultivated mycelium water/alcohol extracts and cut-log wood cultivated fruiting body water/alcohol extracts for the chemotherapy drug 5-FU on inhibiting tumor proliferation.

FIG. 3 shows the recovery of reduced CFU-GM colony number in the bone marrow of 5-FU treated mice by co-administrating with the A. cinnamomea composition (L) of solid-state cultivated mycelium water/alcohol extracts and cut-log wood cultivated fruiting body water/alcohol extracts.

FIG. 4 shows the attenuation of the side effects of the chemotherapy drug 5-FU on reducing numbers of white blood cells (in FIG. 4A), red blood cells (in FIG. 4B), lymphocytes (in FIG. 4C) and neutrophils (in FIG. 4D) by the A. cinnamomea composition (L) of solid-state cultivated mycelium water/alcohol extract and cut-log wood cultivated fruiting body water/alcohol extract.

FIG. 5 shows the improvement of marrow inhibition (reduced CFU-GM colony number) caused by the chemotherapy drugs 5-FU and oxaliplatin in the combination of the A. cinnamomea composition (L) of solid-state cultivated mycelium water/alcohol extract and cut-log wood cultivated fruiting body water/alcohol extracts. *, compared to control group, p<0.05; #, compared to 5-FU+oxa group, p<0.05.

FIG. 6 shows the recovery of reduced white blood cell number caused by the chemotherapy drugs 5-FU and oxaliplatin in the combination of the A. cinnamomea composition (L) of solid-state cultivated mycelium water/alcohol extract and cut-log wood cultivated fruiting body water/alcohol extracts. *, compared to control group, p<0.05; #, compared to 5-FU+oxa group, p<0.05.

FIG. 7 shows the attenuation of the red blood cell reduction caused by the chemotherapy drugs 5-FU and oxaliplatin in the combination of the A. cinnamomea composition (L) of solid-state cultivated mycelium water/alcohol extracts and cut-log wood cultivated fruiting body water/alcohol extracts. *, compared to control group, p<0.05; #, compared to 5-FU+oxa group, p<0.05.

### DETAILED DESCRIPTION OF THE INVENTION

The other characteristics and advantages of the present invention will be further illustrated and described in the following examples. The examples described herein are using for illustrations, not for limitations of the invention.

### Example 1. Preparation of the Antrodia cinnamomea Composition

Firstly, the water/alcohol extracts of cut-log wood cultivated fruiting body of A. cinnamomea was prepared as follow: the fresh cut-log wood cultivated fruiting body of A. cinnamomea was dried at a low temperature of 45° C. in an oven for two days. The dried and powdered fruiting body was extracted in 10× volume of 95% ethanol (w:v=1:10) with sonication for 30 min and further immersion extracted overnight.

The extract was filtered through No.1 filter paper with suction, and the filtered extraction residue was subjected to the 95% ethanol extraction and filtration steps as described above to obtain an ethanol extract. The filtered extraction residue was subjected to a water extraction filtration step by boiling in 10× volume of water (w:v=1:10) for two hours. Repeated the water extraction filtration step for two times to obtain a water extract. The ethanol extract and water extract were combined and concentrated to obtain an A. cinnamomea cut-log wood cultivated fruiting body water/alcohol extracts (FB).

The water/alcohol extracts of solid-state cultivated mycelium of A. cinnamomea was prepared as follow: the dried and powdered solid-state cultivated mycelium was extracted in 10× volume of 95% ethanol (w:v=1:10) with sonication for 60 min and further immersion extracted overnight.

The extract was filtered through No.1 filter paper with suction, and the filtered extraction residue was repeatedly subj ected to the 95% ethanol extraction and filtration steps for two times to obtain an ethanol extract. The filtered extraction residue was subjected to a water extraction filtration step by boiling in 10× volume of water (w:v=1:10) for two hours. Repeated the water extraction filtration step for two times to obtain a water extract. The ethanol extract and water extract were combined and concentrated to obtain an A. cinnamomea solid-state cultivated mycelium water/alcohol extracts (SC).

The A. cinnamomea compositions 40% FB/SC, 20% FB/SC, 10% FB/SC and 5% FB/SC were prepared by combining the obtained cut-log wood cultivated fruiting body water/alcohol extracts (FB) and the obtained solid-state cultivated mycelium water/alcohol extracts (SC) at the ratio of 40% FB/60% SC, 20% FB/80% SC, 10% FB/90% SC and 5% FB/95% SC (% W/W), respectively.

### Example 2. Effect of the Antrodia cinnamomea Composition on Inhibiting Tumor Cell Proliferation

The 50 mg/mL solutions of obtained solid-state cultivated mycelium water/alcohol extracts (SC) and wood cut-log cultivated fruiting body water/alcohol extracts (FB) in Example 1 were prepared in 100% DMSO. The 50 mg/mL stock solution of A. cinnamomea compositions used in this example were prepared by mixing the solid-state cultivated mycelium water/alcohol extracts and cut-log wood cultivated fruiting body water/alcohol extracts at the ratio of 100% FB, 40% FB/60% SC, 20% FB/80% SC, 10% FB/90% SC, 5% FB/95% SC and 100% SC (W/W), respectively. The A. cinnamomea compositions were diluted to 8 solutions of 40, 30, 20, 10, 5, 1, 0.5 and 0.25 mg/mL with DMSO, then further 20-fold diluted with cell culture medium containing 5% FBS to the concentration of 2,000, 1,500, 1,000, 500, 250, 50, 25, 12.5 μg/mL, and the final concentrations of 200, 150, 100, 50, 25, 5, 2., 1.25 μg/mL in each well of 96-well plate, respectively. The 5-FU treated group is used as a positive control.

Eight cancer cell lines including A549 (lung cancer), NCI-H460 (lung cancer), SW480 (colon cancer), Colo205 (colon cancer), MKN45 (gastric cancer), AGS (gastric cancer), MDA-MB-231 (breast cancer) and MCF-7 (breast cancer) were inoculated in a 96-well plate at the density of 6×10³ cells/well, and cultured with 180 μL/well of culture medium at 37° C. for 4 hrs. 20 μL of 5% FBS culture medium containing various concentrations of the A. cinnamomea compositions were added to the cultured cells, and incubated at 37° C. for 48 hrs. The culture medium was removed, the 5% FBS culture medium containing MTS was added, and incubated at 37° C. for 1 hr. Then the absorbance value was read at a wave length of 490 nm on an ELISA Reader. The IC₅₀ was calculated by using the program GraphPad Prism 5. The data were listed in Table 1.

TABLE 1
Effects of A. cinnamomea compositions with various ratio of the solid-state
cultivated mycelium water/alcohol extracts and cut-log wood cultivated fruiting body
water/alcohol extracts on the inhibition of cell proliferation in eight cancer cell lines

| | 100% | 40% | 20% | 10% | 5% | 100% |
|---|---|---|---|---|---|---|
| Cell | FB | FB/SC | FB/SC | FB/SC | FB/SC | SC |
| A549 | 175.0 | 165.2 | 176.6 | >200 | 189.7 | 189.2 |
| NCI-H460 | 157.4 | 139.3 | 142.6 | 155.6 | 158.5 | >200 |
| AGS | 133.0 | 145.5 | 144.5 | 144.5 | 137.7 | 137.1 |
| MKN45 | 69.7 | 105.2 | 104.0 | 104.2 | 103.8 | 108.9 |
| SW480 | 111.2 | 130.3 | 151.7 | 152.8 | 159.6 | 157.4 |
| COLO 205 | 99.8 | 131.5 | 167.9 | 173.4 | 182.0 | 187.9 |
| MDA-MB-231 | 104.2 | 150.7 | 143.5 | 137.7 | 138.4 | 141.9 |
| MCF-7 | 98.2 | 166.0 | 155.2 | 157.0 | 150.7 | 155.2 |

As shown in Table 1, the best inhibitory effects on the proliferation of lung cancer cells A549 and NCI-H460 were provided by the A. cinnamomea composition of 40% FB/SC, which showed a better proliferation inhibiting effect than the FB group. The best inhibitory effects on breast and colon cancer cell proliferation were showed in the 100% FB group. In the test, it is found that 100% SC was most effective for gastric cancer cells when compared to the lung, colon and breast cancer cells, the IC₅₀ for AGS cell is 137.1 μm/mL and for MKN45 cell is 108.9 μg/mL. The most sensitive cancer cell to the treatments of A. cinnamomea composition is MKN45 (gastric cancer) cell, with IC₅₀ in a range of 69.7˜108.9 μm/mL.

### Example 3. Effect of the Antrodia cinnamomea Composition on Inhibiting Gastric Tumor Growth In Vivo

In this example, the effects of the A. cinnamomea composition of solid-state cultivated mycelium water/alcohol extracts and cut-log wood cultivated fruiting body water/alcohol extracts on gastric tumor growth were further evaluated in an animal model. The test A. cinnamomea compositions included 5% FB/95% Sc, 10% FB/90% SC, 20% FB/80% SC, and 40% FB/60% SC, respectively. The animal used in the experiment is an immune deficient mouse (nude mice) implanted MKN45 gastric cancer cells. 10 days after the cancer cell implantation, the animals were orally given the tested A. cinnamomea compositions (300 mg/kg/day) by oral gavage for 21 days. The treated animals were scarified. The tumor was isolated and weighted, as the key indicator for assessing the inhibition of tumor growth.

The implantation of MKN45 cells caused weight loss of about 3 g in nude mice, but no weight loss was observed in the A. cinnamomea composition treated mice, and no significant difference in body weight when compared to the normal control or 5-FU group. It is indicated that the test A. cinnamomea compositions will not increase the amplitude of weight decrease in the tumor-bearing mice. After administrating for 3 weeks, animal were scarified. The tumor was isolated and weighted to confirm the significant decrease in tumor weight by 5-FU injection and the A. cinnamomea composition of 10% FB/90% SC, with P value of 0.001 and 0.046, respectively.

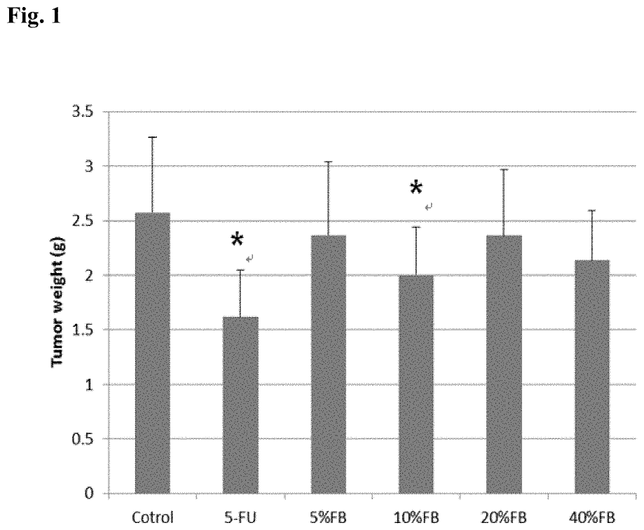

Additionally, as shown in FIG. 1, the A. cinnamomea composition of 10% FB/90% SC was demonstrated to reduce tumor weight with no significant effect on the weight loss of tumor loading mice, and the gastric tumor inhibiting effect of the A. cinnamomea composition of 10% FB/90% SC was similar to the effect of the A. cinnamomea composition of 40% FB/60% SC.

### Example 4. Enhanced Inhibiting Effects on Cancer Cell Proliferation of Chemotherapy Drugs by the Antrodia cinnamomea Composition

The effects of the A. cinnamomea composition individually or combined with chemotherapy drugs on the inhibition of gastric cell proliferation were tested in MKN45, AGS and HGC27 cell lines. The preparation of test drugs including A. cinnamomea cut-log wood cultivated fruiting body water/alcohol extracts (FB), A. cinnamomea solid-state cultivated mycelium water/alcohol extracts (SC) and their combination (FB+SC), and chemotherapy drugs including 5-FU and Epirubicin in the combined treatments shall reference the IC₂₅ concentration of the test drug and the chemotherapy drug. The groups containing only a test drug or a chemotherapy drug of its IC₂₅ are reference index for the stability of the experiment. The initial synergism concentration of the test drug and chemotherapy drug was the combination of their individual IC₂₅, and the half dilution was performed sequentially from the concentration.

The MKN45 and AGS cells were cultured in RPMI-1640 medium containing 5% FBS, and HGC27 cell was cultured in MEM medium containing 5% FBS. 6×10³ cells were inoculated into each well of a 96-well plate with 180 μL of culture medium. Cells were cultured at 37° C. for 4 hrs, and then 20 μL of test drug was added in triplicate of each concentration. The culture medium was removed after cultured at 37° C. for 48 hrs. The 5% FBS medium containing MTS was added and incubated at 37° C. for 1 hr. The absorbance at 490 nm was measured by an ELISA Reader. The IC₂₅, IC₅₀, IC₇₅ and combination index (CI) were calculated by using the program GraphPad Prism 5. The data were listed in Table 2.

TABLE 2
Synergism combination index (CI) of the A. cinnamomea
composition with 5-FU and Epirubicin (Epi)

| | Test drug | | | | | |
|---|---|---|---|---|---|---|
| Chemotherapy | FB | | SC | | FB + SC | |
| drug | 5-FU | Epi | 5-FU | Epi | 5-FU | Epi |
| MKN45 | 1.10 | 0.60 | 1.71 | 1.02 | 0.98 | 0.77 |
| AGS | 1.30 | 1.20 | 1.05 | 1.05 | 1.17 | 1.16 |
| HGC27 | 1.09 | 1.69 | 2.21 | 0.92 | 1.09 | 1.21 |

The synergism of the A. cinnamomea composition combined with chemotherapy drugs 5-FU and Epirubicin was judged by the combination index (CI). The concentration of individual drugs inhibiting 25% of cell activity in the combined treatment were obtained by the interpolation method, and the obtained concentrations were divided by the original IC₂₅ value of individual drugs respectively, and then the sum of two divided values was the CI value. Theoretically, it is considered as additive effect when the CI is equal to 1, and considered as synergism when the CI is less than 1. From the data shown in Table 2, the treatment of FB or FB+SC combined with Epirubicin on MKN45 cells showed a synergistic effect, with the CI value of 0.60 and 0.77 respectively. The treatment of SC combined with Epirubicin showed an additive effect, with the CI value of 1.02. The treatment of FB+SC combined with 5-FU also showed a synergistic effect, with the CI value of 0.98. In the AGS and HGC27 cells, the CI values of the combined treatment of 5-FU or Epirubicin with the three individual test drugs were approaching or greater than 1. The treatment of FB or FB+SC combined with 5-FU showed an additive effect. The results suggested that the Antrodia cinnamomea composition of present invention exhibits effect on promoting the inhibition of cancer cell proliferation of chemotherapy drugs.

### Example 5. Enhanced Inhibiting Effects on Tumor Growth of Chemotherapy Drugs In Vivo by the Antrodia cinnamomea Composition

In this example, the auxiliary effect of the A. cinnamomea composition on the anti-tumor agent 5-FU was confirmed in an animal model. Balb/c nu/nu mice of six-week old, purchased from the National Laboratory Animal Center, were used in the test. The 5-FU (25 mg/kg, ip) treated group was used as positive group, with drug administration frequency of three times a week. The A. cinnamomea composition (L) of solid-state cultivated mycelium water/alcohol extract and cut-log wood cultivated fruiting body water/alcohol extract was orally administered to mice once a day with following dosages: 1× dose of 680 mg/kg, 0.5× dose of 340 mg/kg, 0.25× dose of 170 mg/kg and 0.125× dose of 85 mg/kg. If the mice had been injected with 5-FU, the L was orally treated four hours after the application of 5-FU. Human gastric cancer MKN-45 cells (3×10⁶) were implanted into immune deficient nude mice, and the drug administration was started when the tumor size reached to 100˜200 mm³. The tested animal groups included the control, 5-FU, SFU+L, 5-FU+0.5L, 5-FU+0.25L and 5-FU+0.125L groups, with 8 mice in each group.

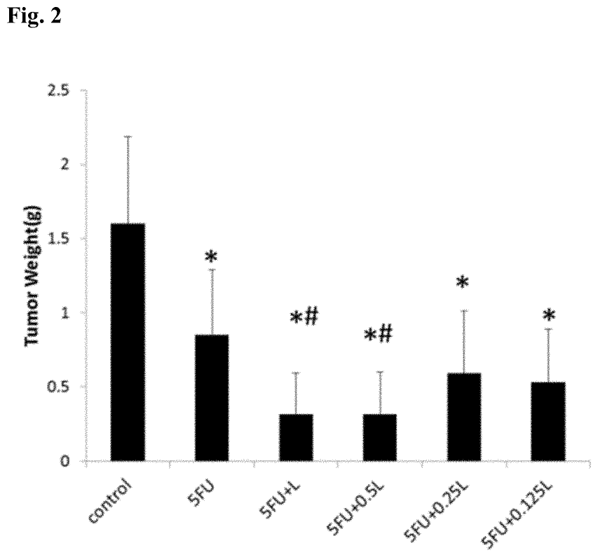

As shown in FIG. 2, the treatment of 5-FU alone significantly inhibited the tumor development when compared to the control, wherein the tumor weight in control was 1.06 g and the tumor weight in the 5-FU treated group was 0.85 g, p<0.05. The tumor inhibiting effect was significantly enhanced in the 5-FU+L and 5-FU+0.5L groups when compared to the 5-FU alone group, wherein the tumor weight in the 5-FU treated group was 0.85 g, while the tumor weight in the 5-FU+L group was 0.32 g and the tumor weight in the 5-FU+0.5L group was 0.32 g, p<0.05. The tumor inhibiting effect in 5-FU+0.25L treated group (with tumor weight of 0.59) and 5-FU+0.125L treated group (with tumor weight of 0.53) were better than the 5-FU treated group, although not reaching statistical significance.

### Example 6. Recovery Effects the Antrodia cinnamomea Composition on Blood Cell Reduction Caused by Chemotherapy Drug 5-FU

The effect of the A. cinnamomea composition (L) on recovering the blood cell reducing caused by the application of chemotherapy drug 5-FU was evaluated in the colony test of CFU-GM in bone marrow. C57BL/6 mice of eight-week old, purchased from the National Laboratory Animal Center, were used in the test. The tested animal groups included the control, 5-FU, SFU+L, 5-FU+0.5L, 5-FU+0.25L and 5-FU+Angiotensin II (Aii) groups, with 6 mice in each group. After the intraperitoneal injection of 5-FU (200 mg/kg) at first day, the test drug A. cinnamomea composition (L) was orally administered to the animals, or the positive control drug Angiotensin II (Aii, 100 μg/kg) was intraperitoneally injected to the animals at day 4˜10. Dosages of the A. cinnamomea composition (L) were used as follow: 1× dose of 680 mg/kg, 0.5× dose of 340 mg/kg and 0.25× dose of 170 mg/kg. Whole blood was collected from cheek before sacrificing the animals at day 11, and the Complete Blood Count (CBC) was performed to record the number of blood cells, including white blood cells, red blood cells, lymphocytes and neutrophils in peripheral blood. The femur was removed from the sacrificed mouse at day 11 of the experiment. The bone marrow cells were collected for the cultivation of CFU-GM. Cells was cultured for 7 days, and the number of CFU-GM colonies was counted.

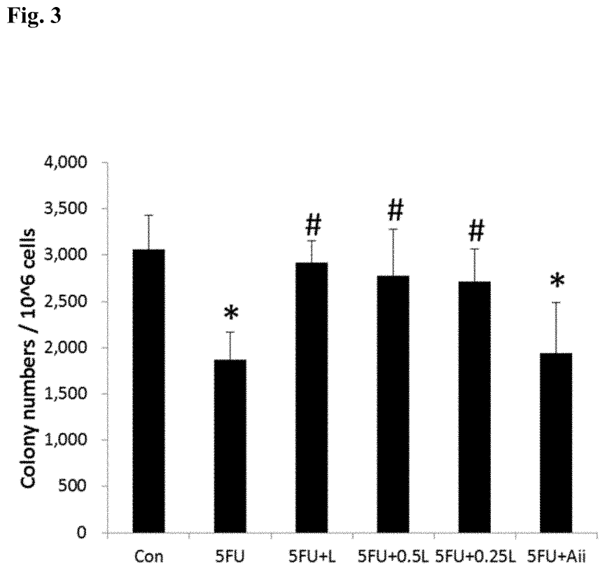

As shown in FIG. 3, the treatment of 5-FU alone significantly reduced the number of CFU-GM when compared to the control, wherein the colony number in the control group was 3,061 colonies/10⁶ marrow cells and the colony number in the 5-FU treated group was 1,869 colonies/10⁶ marrow cells, p<0.05. The combined treatments of 5-FU with the A. cinnamomea compositions (L, 0.5L and 0.25L) significantly increased the CFU-GM colony number when compared to the 5-FU alone group, wherein the colony number in the 5-FU+L group was 2,917 colonies/10⁶ marrow cells, the colony number in the 5-FU+0.5L group was 2,778 colonies/10⁶ marrow cells, and the colony number in the 5-FU+0.25L group was 2,717 colonies/10⁶ marrow cells, p<0.05. The data indicated that the A. cinnamomea composition of present invention could effectively recover the bone marrow suppression caused by 5-FU.

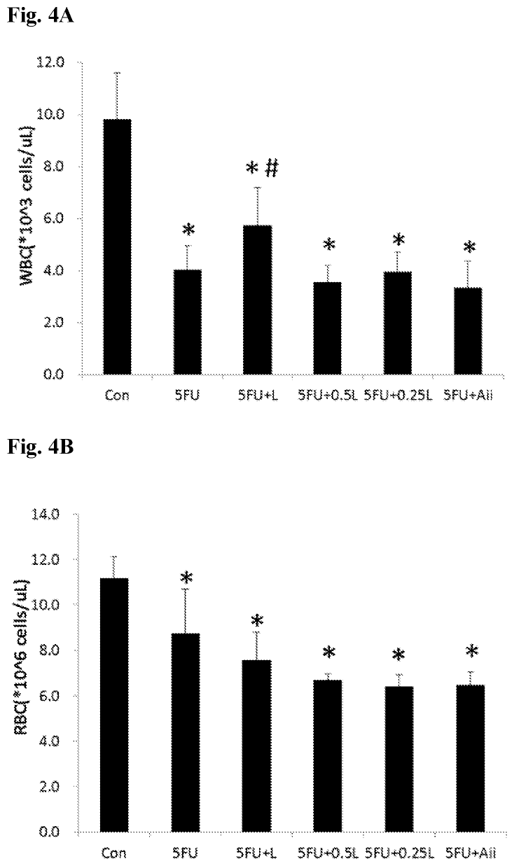

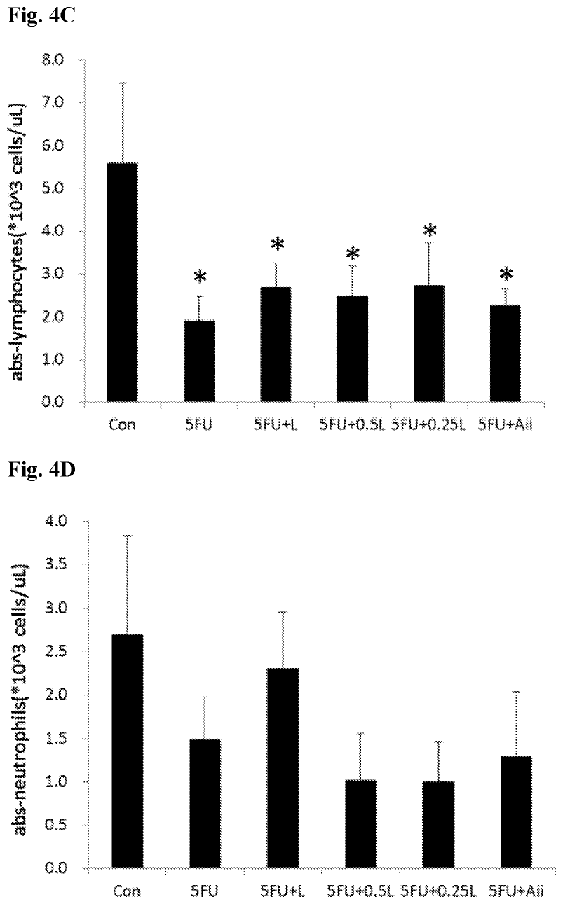

In the results of Complete Blood Count as shown in FIG. 4, the treatment of 5-FU alone significantly reduced the number of white blood cells, red blood cells, lymphocytes and neutrophils when compared to the control, p<0.05. The numbers of white blood cell and lymphocyte were significantly raised in the 5-FU+L group when compared to the 5-FU alone group, p<0.05. The number of neutrophil was also restored, although not reaching the statistical significance. The data indicated that the A. cinnamomea composition of present invention could effectively restore the blood cell reduction caused by 5-FU.

### Example 7. Recovery Effects the Antrodia cinnamomea Composition on the Bone Marrow Suppression and Blood Cell Reduction Caused by Chemotherapy Drug Oxaliplatin

The effect of the A. cinnamomea composition (L) on improving the bone marrow suppression caused by the chemotherapy drug 5-FU and oxaliplatin was evaluated in the animal experiment of evaluating hematopoietic capacity of immune-related cells (colony count of CFU-GM in bone marrow). Male C57BL/6 mice of eight-week old, purchased from the National Laboratory Animal Center, were used in the experiment. The chemotherapy drugs 5-FU and oxaliplatin (oxa) were intraperitoneally injected of at day 0, 2 and 4, with the total dosage of 5-FU being 100 mg/kg and of oxaliplatin being 3 mg/kg. The test drug A. cinnamomea composition (L) was orally administered to the animals once a day at the day 4 9. The daily dose of the A. cinnamomea composition (L) was 510 mg/kg, 340 mg/kg, 227 mg/kg or 151 mg/kg. The tested animal groups included the control, 5-FU+oxa, 5FU+oxa+L510, 5-FU+oxa+L340, 5-FU+oxa+L227 and 5-FU+oxa+L151 groups, with 6 mice in each group. Whole blood was collected from cheek before sacrificing the animals at day 11, and the Complete Blood Count (CBC) was performed to record the number of blood cells, including white blood cells, red blood cells, lymphocytes and neutrophils in peripheral blood. The femur was removed from the sacrificed mouse at day 11 of the experiment. The bone marrow cells were collected for the cultivation of CFU-GM. The cells were cultured for 7 days, and the number of CFU-GM colonies was counted under microscope.

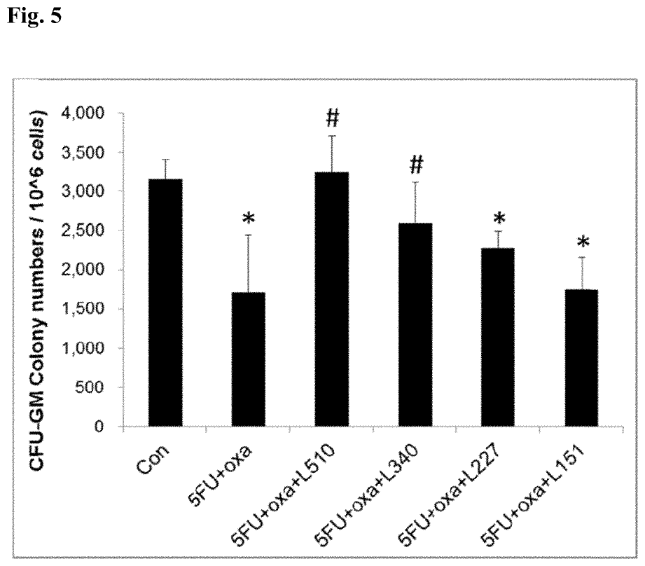

As shown in FIG. 5, the colony number in the control group was 3,153 colonies/10⁶ marrow cells, while the colony number in the 5-FU+oxa treatment group was 1,711 colonies/10⁶ marrow cells (p<0.05), indicating that the treatment of 5-FU+oxa significantly induced the bone marrow suppression. The colony number in the combined treatment of 5-FU+oxa with 510 mg/kg of the A. cinnamomea composition (L) was restored to 3,253 colonies/10⁶ marrow cells, which is competitive to the control. The colony number in the combined treatment of 5-FU+oxa with 340 mg/kg of the A. cinnamomea composition (L) was 2,586 colonies/10⁶ marrow cells, indicating that combination of 5-FU+oxa with 510 mg/kg or 340 mg/kg of the Antrodia cinnamomea composition (L) significantly increased the CFU-GM colony number and in a dose-dependent way. The colony number in combinations of 5-FU+oxa with lower doses (227 mg/kg and 151 mg/kg) of the A. cinnamomea composition (L) were also gradually increased as the raising dose of the A. cinnamomea composition (L). By the results shown in the hematopoietic capacity of immune-related cells, it is suggested that the A. cinnamomea composition of present invention could dose-dependently recover the bone marrow suppression caused by 5-FU+oxa.

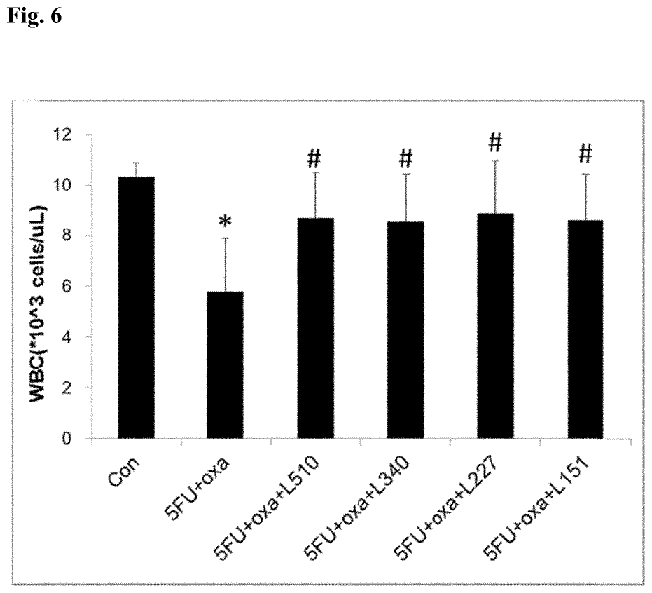

FIG. 6 showed the white blood cell count before the mice sacrifice, indicating decrease of white blood cell number in peripheral blood caused by 5-FU+oxa. The combined treatment of 5-FU+oxa with the A. cinnamomea composition (L) (doses of 510, 340, 227 and 151 mg/kg) significantly raised the number of white blood cell when compared to the 5-FU+oxa treatment group, indicating that the A. cinnamomea composition of present invention could effectively recover the lowering of white blood cell number caused by 5-FU+oxa treatment.

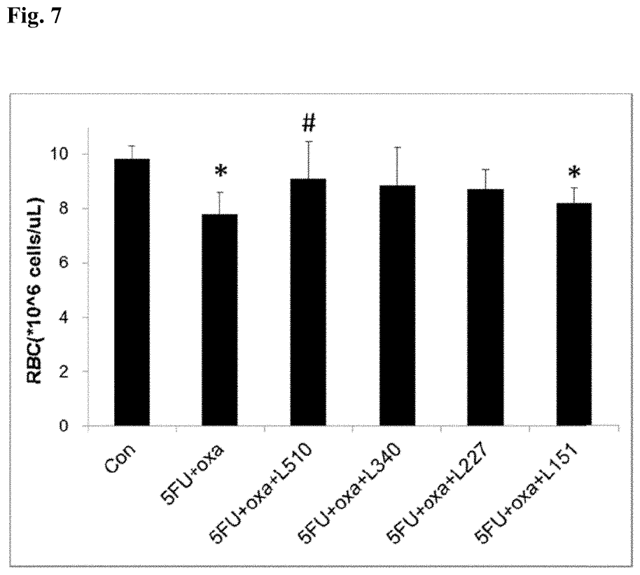

Furthermore, results in FIG. 7 also showed that 5-FU+oxa treatment induced decrease of red blood cell number in peripheral blood. The combinations of 5-FU+oxa with the A. cinnamomea composition (L) could increase the red blood cell number dose-dependently. Especially in the combination with 510 mg/kg of the A. cinnamomea composition (L), the red blood cell number was restored significantly, indicating the red blood cell reduction caused by 5-FU+oxa could be effectively recovered by the action of A. cinnamomea composition of present invention.

To be summarized by the results described above, the A. cinnamomea composition consisted of solid-state cultivated mycelium water/alcohol extracts and cut-log wood cultivated fruiting body water/alcohol extracts exhibits effective functions of improving the anti-cancer and tumor cell inhibition effects of chemotherapy drugs, and reducing and recovering the bone marrow suppression and blood cell reduction caused by chemotherapy treatments. The composition of present invention can be applied to use as an auxiliary for anti-cancer agents, and may significantly decrease the dosage and the side effect of toxic chemotherapy drugs.

1. A method for enhancing the effects of chemotherapy drugs, comprising administering to a subject in need thereof an Antrodia cinnamomea composition, wherein the Antrodia cinnamomea composition is composed of: 60-95% (W/W) of Antrodia cinnamomea solid-state cultivated mycelium water/alcohol extracts and
5-40% (W/W) of cut-log wood cultivated fruiting body water/alcohol extracts.

2. The method of claim 1, wherein the Antrodia cinnamomea composition and the chemotherapy drugs yield an additive or a synergistic effect in treating a cancer.

3. The method of claim 2, wherein the cancer is selected from a group consisted of lung cancer, colon cancer, gastric cancer and breast cancer.

4. The method of claim 2, wherein the cancer is a gastric cancer.

5. The method of claim 1, wherein the Antrodia cinnamomea composition reduces the side effects of chemotherapy drugs.

6. The method of claim 5, wherein the side effect comprising reduction of hematopoietic function in bone marrow.

7. The method of claim 5, wherein the side effect comprising a decrease of blood cells.

8. The method of claim 7, wherein the blood cells are selected from white blood cells, red blood cells, lymphocytes and neutrophils.

9. The method of claim 1, wherein the chemotherapy drug is selected from antimetabolites, alkylating agents, anthracyclines, antibiotics, antimitotic agents, proteasome inhibitors and platinum chemotherapy drugs.

10. The method of claim 1, wherein the chemotherapy drug is 5-fluorouracil (5-FU), epirubicin, oxaliplatin or a combination thereof.
